(19) United States
(12) Patent Application Publication
(10) Pub. No.: US 2016/0279381 A1
(43) Pub. Date: Sep. 29, 2016

(54) NIGHT TERROR LAMP

(71) Applicant: Adam Jude Ahne, Lexington, KY (US)

(72) Inventor: Adam Jude Ahne, Lexington, KY (US)

(21) Appl. No.: 15/043,992

(22) Filed: Feb. 15, 2016

Related U.S. Application Data
(60) Provisional application No. 62/136,806, filed on Mar. 23, 2015.

(51) Int. Cl. A61M 21/02; H05B 37/02

(52) U.S. Cl. CPC A61M 21/02; H05B 37/0227; H05B 37/0272; A61M 2021/0027

(57) ABSTRACT

A lamp turns on a bright light when a sleeper's pulse races during a night terror. The night terror lamp communicates wirelessly with a heart-rate monitor worn by the sleeper. The bright light helps to wake the sleeper and helps to alert others that the sleeper is having a night terror so they can help the sleeper. Other devices and methods are disclosed.

### CROSS REFERENCE TO RELATE APPLICATION

The present application claims the benefit of U.S. Provisional Patent Application No. 62/136,806, filed Mar. 23, 2015, titled “NIGHT TERROR SENTINEL”, the disclosures of which is hereby incorporated by reference.

### FIELD

The present disclosure relates to sleep monitors and more specifically to night terror sleep monitors that provide stimulus to waken the sleeper during a night terror.

### BACKGROUND

This section provides background information related to the present disclosure which is not necessarily prior art.

Night terrors are intense nightmares that occur during non-REM sleep. Night terrors are more common in pre-adolescent children than in adults yet some adults have night terrors especially PTSD sufferers. Night terrors can result in injury to the sleeper because sleepers may move violently as well as injure their vocal cords by screaming. It is difficult to awaken someone having a night terror using sound or touch.

It is known to monitor a heart rate monitor using a cell phone and activate an audible alert when the sleeper's heart rate exceeds an absolute threshold during a night terror. This system has a number of disadvantages. It wakens the sleeper using noise which may not be as effective as turning on a bright light. It requires a cell phone which must be plugged into AC power and unplugged each day, potentially wearing out the connector. A cell phone may be difficult to manipulate when sleepy. Preadolescent children commonly have night terrors and a cell phone may not be an appropriate monitoring device for children because cell phones are fragile, difficult to operate when sleepy, and parents may not want a cell phone in the child's room because the child may play games on the phone instead of sleeping.

### SUMMARY

This section provides a general summary of the disclosure, and is not a comprehensive disclosure of its full scope or all of its features.

According to one example embodiment of the present disclosure, a system for managing night terrors having a heart rate monitor configured to take a heart rate measurement of a user; a lamp; a button; and a controller coupled to the heart rate monitor, the lamp, and the button. The controller is configured to turn on the lamp when the button is pressed while the lamp is off, to turn off the lamp when the button is pressed while the lamp is on, determine whether the heart rate measurement exceeds a threshold, and upon a positive determination turn on the lamp unless less than a disable period has elapsed since the button was last pressed. The lamp consumes at least 0.3 watts when on and the disable period is at least ten minutes.

According to another example embodiment of the present disclosure, an apparatus for managing night terrors having a USB host receptacle having a power connection and a ground connection; a switch coupled to one of the power connection and the ground connection; a radio configured to communicate wirelessly with a heart rate monitor; and a controller coupled to the radio and to the switch. The controller is configured to receive a heart rate measurement from the radio and to close the switch when the heart rate measurement exceeds a threshold.

According to yet another example embodiment of the present disclosure, a system for managing night terrors having a heart rate monitor configured to take a heart rate measurement of a user; a lamp; a button; and a controller configured to receive the heart rate measurement and to turn on the lamp when the heart rate measurement exceeds a threshold and to turn off the lamp when the button is pressed. The threshold is at least one hundred beats per minute.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 shows a diagram of a night terror monitoring system according to one aspect of the present disclosure.

FIG. 2 shows a block diagram of a night terror sentinel according to one aspect of the present disclosure.

FIG. 3 shows a graph of a sleeper's heart rate according to one aspect of the present disclosure.

FIG. 4 shows a method of operating a night terror lamp according to one aspect of the present disclosure.

Corresponding reference numerals indicate corresponding parts throughout the several views of the drawings.

### DETAILED DESCRIPTION

Example embodiments will now be described more fully with reference to the accompanying drawings.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a”, “an” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on”, “engaged to”, “connected to” or “coupled to” another element or layer, it may be directly on, engaged, connected or coupled to the other element or layer, or intervening elements or layers may be present. In contrast, when an element is referred to as being “directly on,” “directly engaged to”, “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath”, “below”, “lower”, “above”, “upper” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

One embodiment of the disclosed system turns on a bright light when a sleeper's heart rate quickens during a night terror. The light may abort the night terror before the sleeper screams. It may also awaken the sleeper's spouse, parent, or caregiver so they can help the sleeper. The disclosed system may also help sufferers of other sleep disorders such as nightmares.

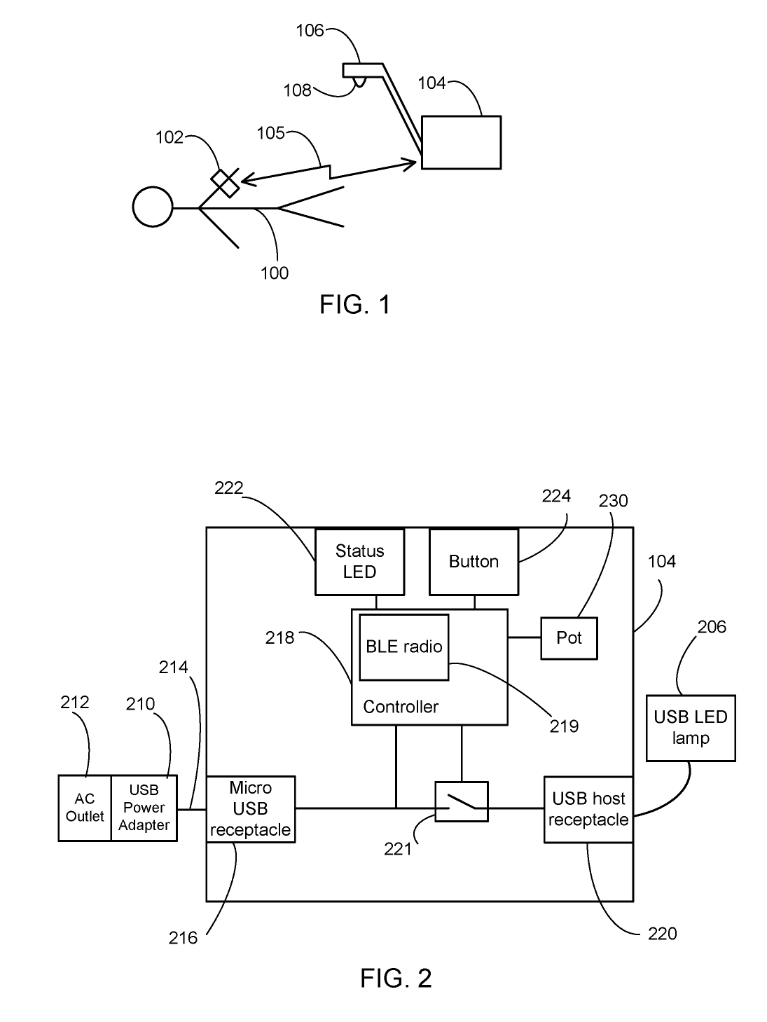

FIG. 1 shows a night terror monitoring system. A sleeper 100 wears a heart rate monitor 102 on their wrist using, for example, a wrist strap. The heart rate monitor takes measurements of the sleeper's heart rate. For example, the heart rate monitor 102 may be a consumer fitness device such as the Scosche RHYTHM+™. Alternatively, other heart rate monitors may be used such as a chest strap, an electrocardiogram connected to electrodes contacting the sleeper, etc. However, a wrist heart rate monitor is preferred because it is the most comfortable for sleeping. The heart rate monitor 102 communicates with a night terror sentinel 104 (NTS) using a wireless transceiver over a wireless communication connection 105 such as, for example, Bluetooth low energy (Bluetooth LE) with the NTS operating in the central/master mode and the heart rate monitor operating in the peripheral/slave mode. Alternatively, other wireless communication systems may be used such as ANT+ designed by the ANT+ Alliance, infrared light, etc. Preferably, the wireless communication connection 105 is bi-directional so the NTS 104 may use the same communication hardware to communicate with, for example, a smart phone to set NTS parameters. While communicating with a smart phone, the NTS may operate in the peripheral/slave mode. It is preferable that the heart rate monitor 102 is not tethered to the NTS 104 by a cable because such a cable would interfere with the sleeper's movement and may make it difficult for the sleeper to go to sleep or remain asleep. Also, if the sleeper 100 is a child, a cable may become wrapped around their neck and cause difficulty breathing. The NTS 104 may disconnect from the heart rate monitor 102 when the sleeper walks out of range of the wireless communication connection 105 and reconnect when the sleeper returns. Preferably, the NTS 104 automatically detects and establishes communication with the heart rate monitor 102 for ease of use. If multiple heart rate monitors are in range, the NTS may automatically select the heart rate monitor with the strongest radio signal since it is probably the nearest. Alternatively, the NTS 104 may remember an identification string of a prior connected heart rate monitor and preferably connect with that device.

The NTS 104 has a lamp 106 that has a light emitter 108 such as, for example, a white LED, a fluorescent bulb, an incandescent bulb, etc. LEDs are preferable because the electronics to switch LEDs are cheaper than the electronics to switch a fluorescent bulb. Also, LEDs are durable which make them suitable for a children's device. White LEDs are preferable over colored LEDs so the user may also use the lamp as a bedside reading lamp. The light emitter 108 is preferably at least as bright as a reading lamp so that the lamp 106 may be used as a reading lamp when the NTS 104 is not being used to monitor a sleeper. Also, a bright lamp will more readily awaken the sleeper than would a dim lamp. The lamp 106 may be integrated into the NTS 104. Preferably, the lamp 106 is detachable from the NTS 104 as described below. Alternatively, the lamp may be mounted in a separate housing and connected wirelessly to the NTS 104. Preferably, the lamp emits at least ten lumens of visible light to wake the sleeper and to provide sufficient illumination for a caregiver to see the sleeper. A white LED lamp that consumes at least 0.3 watts when on provides sufficient illumination.

In operation, the sleeper 100 turns on the heart rate monitor 102 when they go to bed to go to sleep. Preferably, they will leave the NTS 104 powered on all the time. Alternatively, they may power on the NTS 104 as they go to bed to go to sleep. The NTS 104 detects the heart rate monitor 102 and, preferably, turns off the lamp 106 if the lamp 106 is on. The NTS 104 may measure the sleeper's 100 heart rate via the heart rate monitor for a initial period to establish a baseline heart rate. This initial period will be referred to as the baseline period and may be, for example, ten minutes. The baseline period does not begin until the sleeper's heart rate reads greater than zero beats per minute because a reading of zero indicates a problem with the heart rate monitor 102. The NTS 104 may compute an average of the sleeper's 100 heart rate over the final minute of the baseline period and use this computed value as the baseline. After the baseline period, the NTS 104 monitors the sleeper's 100 heart rate and if the heart rate exceeds a threshold the NTS 104 turns on the lamp 106 to waken the sleeper. The threshold is preferably relative to the baseline e.g. fifty percent higher than the baseline. A relative threshold will compensate for resting heart rate variations between sleepers and between the same sleeper on different nights. Alternatively, the threshold may be an absolute threshold e.g. one hundred beats per minute, one hundred ten beats per minute, etc. The threshold may be a combination of a relative threshold with an absolute maximum for example, the lesser of fifty percent higher than the baseline or one hundred fifty beats per minute. The NTS 104 may omit the baseline period if an absolute threshold is used. An increase in the sleeper's heart rate signals that the sleeper is having a night terror, a nightmare, etc. After awakening, the sleeper 100 may turn off the lamp by pressing a button on the NTS 104. The NTS 104 may automatically turn off the lamp once the sleeper's 104 heart rate falls below the threshold. This may prevent waking the sleeper should their heart rate momentarily go above the threshold due to, for example, turning to a new sleeping position.

FIG. 2 shows further details of the NTS 104 and its electrical connections. A USB power adapter 210 is plugged into an AC outlet 212. The USB power adapter 210 supplies, for example, five volts DC at 500 mA max to the NTS 104 though a USB power cable 214 detachably connected to a micro USB receptacle 216. USB power adapters are universally available throughout the world. The NTS 104 contains a controller 218 having a wireless transceiver such as, for example, a Bluetooth LE radio 219 such as, for example, Cypress Semiconductor PSoC 4 Bluetooth Low Energy ASIC. Alternatively, the Bluetooth LE radio may be in a separate integrated circuit coupled to the controller such as, for example, Microchip RN4020. The NTS 104 contains an antenna (not shown) coupled to the Bluetooth LE radio 219 and is preferably a trace on a printed circuit board (PCB) that the controller 218 is mounted to. The controller 218 is connected to a switch 221 such as, for example, a MOSFET transistor, a bi-polar transistor, a relay, etc. The switch 221 connects the voltage at the micro USB receptacle 216 to a USB host receptacle 220 having a power line and a ground line as is known in the art. Preferably, the switch 221 is sized to supply at least 100 mA to the USB host receptacle 220 to power bright USB LED lamps. Preferably, the controller is not connected to USB data lines in the USB host receptacle 220 to avoid exposure to ESD events on these pins. The switch 221 may switch the USB host receptacle 220 power line, ground line, or both. The USB host receptacle 220 is sized to mate to a standard USB type A plug. A USB LED lamp 206 is detachably connected to the USB host receptacle 220 though a standard USB type A plug. The USB LED lamp 206 is, for example, an off-the-shelf device commonly used to illuminate the keys of a laptop keyboard. It is preferable to use a USB host receptacle 220 so a user may select their preferred lamp. The controller 218 is coupled to a status LED 222 and a button 224. The button 224 preferably contains a capacitive touch sensor. Alternatively, the button may contain a tactile mechanical switch. Preferably, the button uses a momentary contact instead of a toggle switch. The button includes a surface the user touches, e.g. a plastic cap, and an electrical component for detecting when the button is actuated, e.g. a tactile mechanical switch.

In operation, the controller 218 activates the switch 221 to turn on the USB LED lamp 206 and deactivates the switch 221 to turn off the USB LED lamp 206. The controller toggles the state of the switch 221 whenever a user presses the button 224. Thus, a user may manually turn the USB LED lamp 206 on or off by pressing the button 224. In this way, the user may use the USB LED lamp 206 as a bedside reading lamp. Also, a sleeper 100 may press the button 224 to turn off the USB LED lamp 206 after the NTS 104 awakens the sleeper 100 from a night terror. After a button press, the controller 218 may disable turning on the switch 221 for a disable period of at least 10 minutes to give time for the user's heart rate to go below the threshold. Preferably, the disable period is at least 20 minutes since it is unlikely that a user will fall back asleep and have a second night terror within 20 minutes of the first night terror. After a button press, the controller 218 may disable turning on the switch 221 until the user's heart rate goes below the threshold or, alternatively, below the threshold plus a guard band. For example, the threshold may be one hundred ten beats per minute (BPM), and the guard band may be ten BPM.

The controller 218 turns on the status LED 222 when a heart rate monitor 102 is not detected. This alerts the sleeper 100 that they need to turn on the heart rate monitor 102. The controller 218 blinks the status LED 222 when the heart rate monitor 102 reports zero heart rate, indicating a problem with the heart rate monitor 102. Once the NTS 104 starts measuring the sleeper's 100 heart rate the controller turns off the status LED 222, as well as the USB LED lamp 206, so the room is dark so the NTS does not make it difficult for the sleeper 100 to sleep. Alternatively, the NTS 104 may turn on the status LED when connected to the heart rate monitor.

The NTS and the lamp may be located in separate rooms when, for example, the sleeper is a child. The lamp may be located in a parent's room so they may wake and help the child during the child's night tenor. The USB LED lamp 206 may be connected to the USB host receptacle 220 through an extension cable. In this way, the lamp may be located in a different room than the NTS.

A radio or other noise making device may be plugged into the USB host receptacle 220 through, for example, a USB to barrel power plug cable. A noise maker may be preferable if the sleeper is blind. Alternatively, the NTS 104 may have a speaker, an audio output connector, etc.

When the sleeper's heart rate exceeds the threshold, the NTS activates the switch 221 and the switch 221 remains activated until either the user touches the button 224 or the sleeper's heart rate falls below the threshold. Alternatively, the NTS may alternate activating the switch 221 continuously for, for example, one minute and activating the switch with a duty cycle of, for example, fifty percent with a two second period for one minute to make the USB LED lamp 206 alternate between steady light and slowly flashing until the user touches the button 224 or the sleeper's heart rate falls below the threshold. The switch activation behavior may be a user selectable parameter. The switch 221 may be protected by a fly back diode in case the USB LED lamp 206 is connected by a long cable with significant inductance.

The heart rate monitor 102 may send a heart rate measurement of the sleeper's heart rate at a rate of, for example, once per second via a wireless transceiver located in the heart rate monitor 102 such as, for example, a Bluetooth LE radio. Preferably, the controller 218 will filter the heart rate measurements by, for example, averaging thirty seconds of heart rate measurements. The controller 218 may use filtered measurements instead of unfiltered measurements when, for example, setting the threshold, determining if the threshold has been exceeded, etc. Filtering helps reduce the variation in the heart measurements caused by the sleeper moving in their sleep.

It is beneficial to locate a wake stimulus, for example a lamp, a distance away from the sleeper so the sleeper does not perceive the stimulus as a threat during a night terror. In contrast, a sleeper may become panicked and hurt themselves if the wake stimulus was incorporated into a wrist-mounted heart rate monitor.

The controller 221 may contain firmware that the controller 221 executes. The firmware configures the controller's behavior. Alternatively, the controller may contain a field programmable gate array (FPGA) or other circuitry that configures the controller's behavior. The controller may contain a combination of firmware and circuitry that configures the controller's behavior.

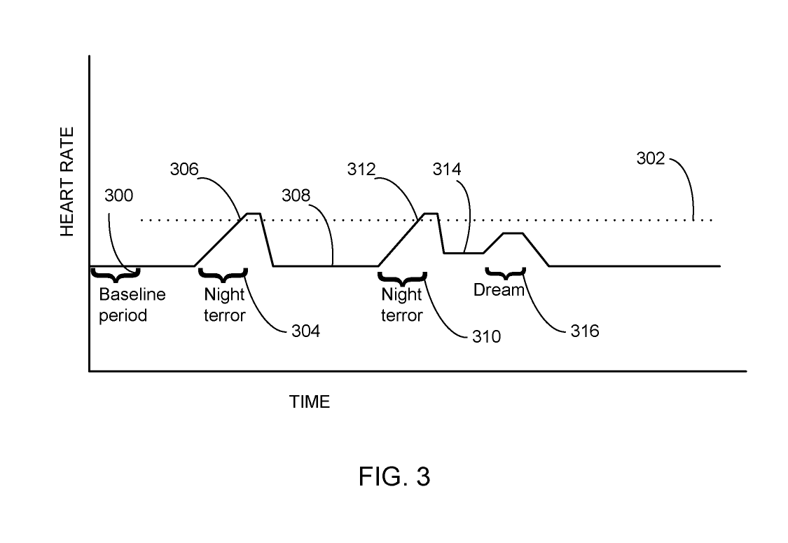

FIG. 3 shows a graph of a sleeper's heart rate during a sleep period. At the end of the initial baseline period 300 the controller 218 measures the sleeper's heart rate to determine a threshold 302 as described above. During a first night terror 304 the sleeper's heart rate increases due to their body's response to the night terror. A first trigger event 306 occurs when the sleeper's heart rate exceeds the threshold for at least a threshold period which is, for example, thirty seconds. This threshold period helps to prevent false trigger events. Alternatively, the threshold period may be omitted. At the first trigger event 306 the controller 218 activates the switch 221 to turn on the USB LED lamp 206. The sleeper wakes, touches the button 224 to turn off the USB LED lamp 206, and their heart rate goes down to a resting rate 308 that is less than the threshold 302. The controller 218 will not reactivate the switch 221 until the user's heart rate goes below the threshold.

Later, a second night terror 310 occurs and the heart rate increases until it exceeds the threshold 302 and a second trigger event 312 occurs. At the second trigger event 312 the controller 218 activates the switch 221 to turn on the USB LED lamp 206. The sleeper wakes, touches the button 224 to turn off the USB LED lamp 206, and their heart rate goes down to a second resting rate 314 that is less than the threshold 302 and more than the previous resting rate 308. Later, the sleeper has a dream and their heart rate increases above the second resting rate 314 but does not exceed the threshold 302. Thus, a trigger event does not occur during the dream 316 and so the controller 218 does not turn on the USB LED lamp 206.

The NTS 104 may have additional features, such as an analog output that is proportional to the sleeper's heart rate for the user to connect to an external data logger. This will help the sleeper understand when their night terrors occur, their maximum heart rate during a night terror, etc. This data may be helpful when setting parameters of the NTS such as, for example, the type of threshold (absolute, relative, both), the threshold value, the duration of the baseline period, etc. A serial UART output may provide the same data logging capability when connected to a serial data logger. The controller may contain non-volatile memory and the controller may store a series of heart rate measurements that may be retrieved using, for example, a Bluetooth LE smart phone. The controller may be configured to detect when the button is pressed continuously for at least one second and when so detected to blink the lamp at a blink rate equal to a heart rate of a prior heart rate measurement such as, for example, the maximum heart rate measured during the preceding session. The user may use this information to adjust the threshold. It is preferable to enable the user to adjust the threshold because resting heart rate and night-terror heart rate may vary from user to user and the adjustment allows the user to personalize the system. Alternatively, a fixed threshold or automatic threshold may be used without a user adjustment.

One or more of the NTS parameters may be adjusted by a user, for example, by communicating with a Bluetooth LE smart phone having a graphic user interface. The NTS parameters may also be adjusted by a user by dip switches inside the NTS, a potentiometer 230, etc. The potentiometer 230 may have a wiper coupled to an ADC in the controller 218, a terminal connected to ground, and a terminal connected to the Vcc of the controller 218. The controller 218 may, for example, set the threshold 302 to 90 beats per minute when the potentiometer is fully counter-clockwise, to 110 beats per minute when the potentiometer is centered, etc. The controller 218 may be connected to USB data lines in the micro USB receptacle 216 to facilitate setting NTS parameters by connecting the NTS 104 to a host computer USB port, to update the NTS firmware, etc. The NTS may have wireless network (e.g. wifi) capability to update NTS firmware, set NTS parameters, control a wifi connected device such as a wifi light bulb, etc. An example NTS parameter is the threshold 302.

The NTS may have an infrared LED port to activate a remote device such as a lamp. The NTS may have a learning mode where it learns the infrared pulse sequence sent by a device remote control so that the NTS may later activate and deactivate the device.

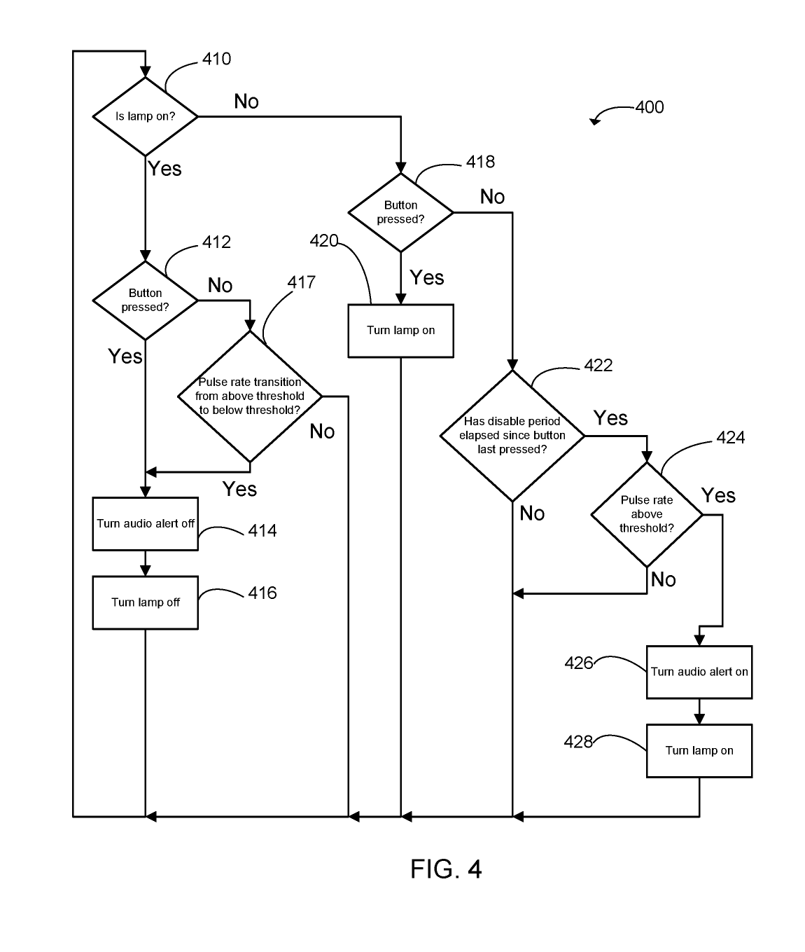

FIG. 4 shows a method of operating a night terror lamp. The controller 218 may be configured to perform the method 400. The method 400 features a minimalistic user interface comprising a single button 224. A user turns the lamp on and off by pressing the button 224. The user also presses the button 224 to turn off the lamp after a night terror. Thus, in the mind of the user the button's function is clear: toggle the state of the lamp i.e. turn the lamp on and off. This minimalistic user interface is beneficial to sleepy users who may be confused or frustrated with a more complicated user interface. The method 400 implements a disable period which prevents the lamp from immediately turning back on when the user presses the button while the user's heart rate is still elevated after a night terror. This would confuse and frustrate the user who wishes to turn off the lamp so they can go back to sleep. The user may want to turn on the lamp to, for example, use it as a bedside reading lamp. Thus, the method 400 allows the user to use the lamp for reading as well as use the lamp to manage night terrors.

At block 410, the controller determines whether the lamp is on. If the lamp is on, at block 412 the controller determines whether the button is pressed. If the button is pressed, at block 414 the controller turns off an audio alert. If the audio alert is already off, or if the system does not feature an audio alert, this block may be omitted. At block 416, the controller turns off the lamp. If the button is not pressed, at block 417 the controller determines whether the user's heart rate has transitioned from above the threshold to below the threshold. If yes, the method continues at block 414. If no, the method repeats starting at block 410. In this way, the lamp will turn off automatically following a temporary spike in the user's heart rate without requiring the user to wake up and turn off the lamp using the button.

If, at block 410, the lamp is not on, at block 418 the controller determines whether the button is pressed. If the button is pressed, at block 420 the controller turns on the lamp. If the button is not pressed, at block 422 the controller determines whether a disable period has elapsed since the button was last pressed. The controller may start a timer whenever the button is pressed and, at block 422, compare the timer to the disable period. Preferably, the disable period is at least ten minutes and more preferably twenty minutes to allow time for the user's heart rate to drop below the threshold following a night terror. If the disable period has not elapsed, the method 400 repeats starting at block 410.

If the disable period has elapsed, at block 424 the controller determines whether the user's heart rate is above the threshold. If it is, at block 426 the controller turns on the audio alert. If the system does not feature an audio alert this block may be omitted. At block 428 the controller turns on the lamp to wake the user. If the user's heart rate is not above the threshold the method repeats starting at block 410.

The NTS is useful to waken an adult sleeper from night terrors. This may shorten the night terror and reduce the severity of the emotional stress and the physical stress of, for example, screaming or moving uncontrollably. The NTS may also awaken a caregiver and provide light so they may give assistance to the sleeper if the sleeper does not waken. The NTS is helpful for children suffers of night terrors. The simple user interface and durable construction make the NTS well suited for children. The NTS may give children a feeling of control over their night terrors. Unlike an audible alert, a lamp alert may avoid waking a sleeping baby in an adjacent room.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

What is claimed is:

1. A system for managing night terrors comprising:
a heart rate monitor configured to take a heart rate measurement of a user;
a lamp;
a button; and
a controller coupled to the heart rate monitor, the lamp, and the button, the controller is configured to turn on the lamp when the button is pressed while the lamp is off, to turn off the lamp when the button is pressed while the lamp is on, determine whether the heart rate measurement exceeds a threshold, and upon a positive determination turn on the lamp unless less than a disable period has elapsed since the button was last pressed,
wherein the lamp consumes at least 0.3 watts when on and the disable period is at least ten minutes.

2. The system of claim 1, wherein the controller is configured to turn off the lamp when the heart rate transitions from above the threshold to below the threshold.

3. The system of claim 1, wherein the heart rate monitor has a wireless transceiver and is coupled to the controller through the wireless transceiver.

4. The system of claim 1, wherein the lamp contains white LEDs.

5. The system of claim 1, further comprising a means for adjusting the threshold by a user.

6. The system of claim 1, wherein the controller is configured to adjust the threshold based on a prior heart rate measurement.

7. The system of claim 1, wherein the lamp emits at least ten lumens of visible light when on.

8. An apparatus for managing night terrors comprising:
a USB host receptacle having a power connection and a ground connection;
a switch coupled to one of the power connection and the ground connection;
a radio configured to communicate wirelessly with a heart rate monitor; and
a controller coupled to the radio and to the switch, the controller is configured to receive a heart rate measurement from the radio and to close the switch when the heart rate measurement exceeds a threshold.

9. The system of claim 8, further comprising a button coupled to the controller, and the controller is configured to close the switch when the button is pressed while the switch is open, and to open the switch when the button is pressed while the switch is closed.

10. The system of claim 8, wherein the radio is a Bluetooth Low Energy device.

11. The system of claim 8, wherein the controller is configured to adjust the threshold based on a prior heart rate measurement.

12. The system of claim 8, further comprising a means for adjusting the threshold by a user.

13. The system of claim 8, further comprising a button coupled to the controller, and the controller is configured to detect when the button is pressed continuously for at least one second and when so detected to blink the lamp at a blink rate equal to a heart rate of a prior heart rate measurement.

14. A system for managing night terrors comprising:
a heart rate monitor configured to take a heart rate measurement of a user;
a lamp;
a button; and
a controller configured to receive the heart rate measurement and to turn on the lamp when the heart rate measurement exceeds a threshold and to turn off the lamp when the button is pressed,
wherein the threshold is at least one hundred beats per minute.

15. The system of claim 14, wherein the lamp emits at least ten lumens of visible light when on.

16. The system of claim 14, wherein the controller is configured to disable turning on the lamp, when the heart rate measurement exceeds a threshold, for at least ten minutes after the button is pressed.

17. The system of claim 14, further comprising a means for adjusting the threshold by a user.

18. The system of claim 14, wherein the controller is configured to adjust the threshold based on a prior heart rate measurement.

19. The system of claim 14, wherein the controller is configured to detect when the button is pressed continuously for at least one second and when so detected to blink the lamp at a blink rate equal to a heart rate of a prior heart rate measurement.

20. The system of claim 14, wherein the controller is configured to turn off the lamp when the heart rate measurement transitions from above the threshold to below the threshold.
